(19) United States
(12) Patent Application Publication
(10) Pub. No.: US 2007/0225299 A1
(43) Pub. Date: Sep. 27, 2007

(54) Novel Combination Containing a Stimulator of Soluble Guanylate Cyclase and a Lipid-Lowering Substance

(75) Inventors: Hilmar Bischoff, Wuppertal (DE); Johannes-Peter Stasch, Solingen (DE); Stefan Weigand, Penzberg (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Healthcare AG, Leverkusen (DE)

(21) Appl. No.: 10/578,412

(22) PCT Filed: Oct. 26, 2004
(86) PCT No.: PCT/EP04/12049

§ 371 Date: Dec. 26, 2006

(30) Foreign Application Priority Data
Nov. 6, 2003 (DE) 10351903.3

(51) Int. Cl. A61K 31/495

(52) U.S. Cl. 514/256

(57) ABSTRACT

A combination product which comprises as pharmaceutically active ingredients at least one active ingredient component A and at least one active ingredient component B, where active ingredient component A is a direct soluble guanylate cyclase stimulator of the formula (I) and active ingredient component B is a lipid-lowering agent, is described and claimed. Methods for treating various conditions using combination therapy are also claimed.

The present invention relates to a novel combination product comprising at least one lipid-lowering agent and at least one stimulator of soluble guanylate cyclase of the formula (I).

One of the most important cellular transmission systems in mammalian cells is cyclic guanosine monophosphate (cGMP). Together with nitric oxide (NO), which is released from the endothelium and transmits hormonal and mechanical signals, it forms the NO/cGMP system. Guanylate cyclases catalyze the biosynthesis of cGMP from guanosine triposphate (GTP). The representatives of this family disclosed to date can be divided both according to structural features and according to the type of ligands into two groups: the particulate guanylate cyclases which can be stimulated by natriuretic peptides, and the soluble guanylate cyclases which can be stimulated by NO. The soluble guanylate cyclases consist of two subunits and very probably contain one heme per heterodimer, which is part of the regulatory site. The latter is of central importance for the mechanism of activation. NO is able to bind to the iron atom of heme and thus markedly increase the activity of the enzyme. CO is also able to attach to the central iron atom of heme, but the stimulation by CO is distinctly less than that by NO.

Through the production of cGMP and the regulation, resulting therefrom, of phosphodiesterases, ion channels and protein kinases, guanylate cyclase plays a crucial part in various physiological processes, in particular in the relaxation and proliferation of smooth muscle cells, in platelet aggregation and adhesion and in neuronal signal transmission, and in disorders caused by an impairment of the aforementioned processes.

Compounds, such as organic nitrates, whose effect is based on the release of NO have to date been exclusively used for the therapeutic stimulation of soluble guanylate cyclase. NO is produced by bioconversion and activates soluble guanylate cyclase by attaching to the central iron atom of heme. Besides the side effects, the development of tolerance is one of the crucial disadvantages of this mode of treatment.

Some substances which directly stimulate soluble guanylate cyclase, i.e. without previous release of NO, have been described in recent years, such as, for example, 3-(5′-hydroxymethyl-2′-furyl)-1-benzylindazole (YC-1, Wu et al., Blood 84 (1994), 4226; Mülsch et al., Br. J. Pharmacol. 120 (1997), 681), fatty acids (Goldberg et al., J. Biol. Chem. 252 (1977), 1279), diphenyliodonium hexafluorophosphate (Pettibone et al., Eur. J. Pharmacol. 116 (1985), 307), isoliquiritigenin (Yu et al., Brit. J. Pharmacol. 114 (1995), 1587) and various substituted pyrazole derivatives (WO 98/16223).

In addition, WO 98/16507, WO 98/23619, WO 00/06567, WO 00/06568, WO 00/06569, WO 00/21954, WO 02/42299, WO 02/42300, WO 02/42301, WO 02/42302, WO 02/092596 and WO 03/004503 describe pyrazolopyridine derivatives as direct stimulators of soluble guanylate cyclase. A combination of pyrazolopyridine derivatives and lipid-lowering agents is described in WO 03/015770.

It has now surprisingly been found that the effect of direct stimulators of soluble guanylate cyclase of the formula (I) 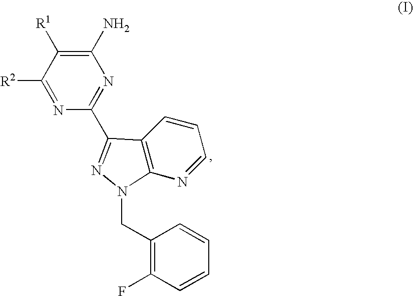
in which
R¹ is —NR³C(═O)OR⁴,
R² is hydrogen or NH₂,
R³ is hydrogen or (C₁-C₄)-alkyl,
R⁴ is (C₁-C₆)-alkyl
and of salts, isomers and hydrates thereof,
can be enhanced on administration of a lipid-lowering agent in combination with these stimulators of soluble guanylate cyclase.

It is possible in this way for example to reduce the amount of direct soluble guanylate cyclase stimulator of the formula (I), or amount of lipid-lowering agent, which are necessary for the treatment in particular of the above-mentioned diseases and thus diminish the potential for side effects.

The present invention thus relates to a combination product comprising
as active ingredient component A at least one direct soluble guanylate cyclase stimulator; and
as active ingredient component B at least one lipid-lowering agent.

The term “combination product” as used for the purposes of the present invention means that the two active ingredient components A and B can be administered either simultaneously or sequentially (i.e. separately from one another).

The term “combination product” encompasses, according to the invention, ingredients A and B either in one functional unit, i.e. as true combination (e.g. as mixture, mix or blend), or else (spatially) separate in juxtaposition, i.e. as so-called kit of parts.

A further aspect of the present invention is a combination therapy for diseases which can be influenced by stimulating soluble guanylate cyclase, in particular the abovementioned diseases, with a combination product which comprises at least one direct stimulator of soluble guanylate cyclase of the formula (I) and at least one lipid-lowering agent.

As mentioned previously, the combination of the invention can be administered, i.e. the combination therapy of the invention can take place, in such a way that the active ingredient components A and B are administered simultaneously or successively. It is possible in this case for the active ingredient components A and B, as described above, to be present either in one functional unit (i.e. as true combination such as, for example, as mixture, mix or blend) or else (spatially) separate in juxtaposition (i.e. as so-called kit or kit-of-parts).

In a preferred embodiment of the present invention, the active ingredient components A and B are administered separately from one another, in particular sequentially.

This can take place for example by administering a daily dose of the lipid-lowering agent some days (e.g. about 1 week or else only 1-4 days) before administration of the direct soluble guanylate cyclase stimulator of the formula (I).

It is also possible to administer the direct soluble guanylate cyclase stimulator of the formula (I) within a pre-existing lipid-lowering agent therapy, for example for patients with severe hypercholesterolemia, in whom the elevated cholesterol levels are already treated permanently with lipid-lowering agents. In this case, therefore, administration of the lipid-lowering agent can also be continued before and in parallel with the administration of the direct soluble guanylate cyclase stimulator.

In a preferred embodiment of the present invention, the active ingredient components A and B of the combination product of the invention are thus administered sequentially, preferably the lipid-lowering agent preceding, i.e. prior to, administration of the direct soluble guanylate cyclase stimulator of the formula (I).

Without wishing in this connection to be bound to a particular theory, the improvement in the effect of the direct soluble guanylate cyclase stimulator of the formula (I) through simultaneous, sequential or parallel administration of lipid-lowering agents can presumably be explained by the fact that the lipid-lowering agents improve the impaired endothelial function by generating nitric oxide (NO) (Current Opinion in Lipidology, 1997, Vol. 8, pages 362-368 and Circulation 1998, 97, pages 1129-1135). It has been possible to show that direct soluble guanylate cyclase stimulators show a synergistic effect in combination with NO (cf., for example, WO 00/06569, FIG. 1).

According to the present invention, the lipid-lowering agent can be selected from the group of:
HMG-CoA reductase inhibitors,
squalene synthase inhibitors,
bile acid absorption inhibitors (also called bile acid anion exchangers or bile acid sequestrants),
fibric acid and its derivatives,
nicotinic acid and its analogs and
ω3-fatty acids.

For further details of the aforementioned lipid-lowering agents, reference is made in this connection to the article by Gilbert R. Thompson & Rissitaza P. Naoumova “New prospects for lipid-lowering drugs” in Exp. Opin. Invest. Drugs (1998), 7(5), pages 715-727, the entire contents of which are hereby expressly incorporated by reference.

The lipid-lowering agents preferred according to the invention amongst those aforementioned are the HMG-CoA reductase inhibitors. The abbreviation “HMG-CoA” in this connection stands for “3-hydroxymethylglutaryl-coenzyme A”.

In turn, the HMG-CoA reductase inhibitors particularly preferred according to the invention belong to the substance class of vastatins—usually referred to only as “statins” for simplicity in the literature.

Those statins which are in turn particularly preferred according to the invention are
atorvastatin (commercially available under the name Lipitor® from Parke-Davis);
cerivastatin (commercially available under the name Lipobay® or Baycol® from Bayer);
fluvastatin (commercially available under the name Lescol® from Novartis);
lovastatin (commercially available under the name Mevacor® from Merck);
pravastatin (commercially available under the name Lipostat® from Bristol-Myers Squibb);
simvastatin (commercially available under the name Zocor® from Merck);
pitavastatin (also called “nisvastatin”; NK-104; systematic name: [S—[R*,S*-(E)]]-7-[2-cyclopropyl-4-(4-fluorophenyl)-3-quinolinyl]-3,5-dihydroxy-6-heptenoic acid);
dalvastatin;
mevastatin;
dihydrocompactin;
compactin; and
rosuvastatin (commercially available under the name Crestor® from AstraZeneca; systematic name: (+)-(3R,5S)bis(7-(4-(4-fluorophenyl)-6-isopropyl-2-(N-methyl-N-methanesulfonylamino)pyrimidin-5-yl) -3,5-dihydroxy-6(E)-heptenoic acid);
   and the respective salts, hydrates, alcoholates, esters and tautomers thereof.

Very particularly preferred among these are atorvastatin, cerivastatin, fluvastatin, lovastatin, pravastatin, pitavastatin, simvastatin and rosuvastatin, and the respective salts, hydrates, alcoholates, esters and tautomers thereof.

Very particularly preferred among these in turn are cerivastatin and atorvastatin and the respective salts, hydrates, alcoholates, esters and tautomers thereof.

For further details of the aforementioned statins, reference is made to the discussions in Drugs of the Future 1994, 19(6), pages 537-541 and 1995, 20(6), page 611 and 1996, 21(6), page 642, the contents of each of which are incorporated in their entirety by reference.

The term “salt” for the purposes of the present invention means in each case physiologically acceptable salts of the respective compounds. These may be, for example: salts with mineral acids, carboxylic acids or sulfonic acids, in particular with hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, citric acid, fumaric acid, maleic acid or benzoic acid or else mixed salts thereof. However salts with conventional bases are also possible, such as, for example, alkali metal salts (e.g. sodium or potassium salts), alkaline earth metal salts, (e.g. calcium or magnesium salts) or ammonium salts derived from ammonia or organic amines such as, for example, ethylamine, diethylamine, triethylamine, diethanolamine, triethanolamine, dicyclohexylamine, dimethylaminoethanol, arginine, lysine or ethylenediamine and mixed salts thereof.

Examples of statin salts which can be used according to the invention are fluindostatin (the monosodium salt of fluvastatin); the monopotassium salt and the calcium salt of pitavastatin; and the calcium salt of (+)-(3R,5S)bis(7-(4-(4-fluorophenyl)-6-isopropyl-2-(N-methyl-N-methanesulfonylamino)pyrimidin-5-yl)-3,5-dihydroxy-6(E)-heptenoic acid (“rosuvastatin”, “ZD 4522” or “S 4522” from Shionogi or AstraZeneca). Further examples of statin salts which can be used according to the invention are the monosodium and monopotassium salts, and the calcium salts of cerivastatin, of atorvastatin and of pravastatin.

Further preferred HMG-CoA reductase inhibitors are described in EP-A-0 325 130 and in EP-A-0-491 226, the contents of which are hereby incorporated by reference. EP-A-0 325 130 relates to substituted pyridines, and EP-A-0-491 226 describes substituted pyridyl-dihydroxyheptenoic acid derivatives and their salts, particularly including cerivastatin which is particularly preferred according to the invention (claim 6 of EP-A-0-491 226).

Likewise preferred according to the invention are the statins mentioned in WO-A-99/11263, the disclosure of which is incorporated by reference.

Equally preferred according to the invention are the HMG-CoA reductase inhibitors mentioned in the publication Bioorganic & Medicinal Chemistry, Vol. 5, No. 2, pages 437-444 (1997), the disclosure of which is hereby incorporated in its entirety by reference.

A further review of HMG-CoA reductase inhibitors is present in Pharmazie in unserer Zeit, Vol. 28, No. 3, pages 147-1152 (1999).

The aforementioned bile acid absorption inhibitors (bile acid sequestrants) which are preferred according to the invention are cholestyramine (commercially available under the name Qestran® from Bristol-Myers Squibb) and colestipol (commercially available under the name Colestid® from Pharmacia & Upjohn) (see also Exp. Opin. Invest. Drugs (1998), 7(5), pages 715-727).

The aforementioned fibric acid derivatives which are preferred according to the invention are ciprofibrate (commercially available under the name Modalim® from Sanofi Winthrop), fenofibrate (commercially available under the name Lipantil® from Fournier), gemfibrozil (commercially available under the name Lopid® from Parke-Davis), bezafibrate and clofibrate (see also Exp. Opin. Invest. Drugs (1998), 7(5), pages 715-727).

Of the aforementioned nicotinic acid analogs, preference is given according to the invention to acipimox (commercially available under the name Olbetam® from Pharmacia & Upjohn) (see also Exp. Opin. Invest. Drugs (1998), 7(5), pages 715-727).

Of the aforementioned ω3-fatty acids, preference is given according to the invention to maxepa (marketed by Seven Seas) (in this connection, see also Exp. Opin. Invest. Drugs (1998), 7(5), pages 715-727).

Direct soluble guanylate cyclase stimulators of the formula (I) which are preferred according to the invention are those in which

R¹ is —NR³C(═O)OR⁴,

R² is hydrogen or NH₂,

R³ is (C₁-C₄)-alkyl,

R⁴ is (C₁-C₄)-alkyl,

and salts, isomers and hydrates thereof.

For the purposes of the present invention, alkyl stands for a linear or branched alkyl radical having usually from 1 to 6, preferably 1 to 4, particularly preferably 1 to 3, carbon atoms, for example and preferably methyl, ethyl, n-propyl, isopropyl, tert-butyl, n-pentyl and n-hexyl.

Direct soluble guanylate cyclase stimulators of the formula (I) which are particularly preferred according to the invention are those in which

R¹ is —NR³C(═O)OR⁴,

R² is NH₂,

R³ is methyl or ethyl,

R⁴ is methyl, ethyl or isopropyl,

and salts, isomers and hydrates thereof.

The direct soluble guanylate cyclase stimulator of the formula (I) which is particularly preferred according to the invention has the following structure: 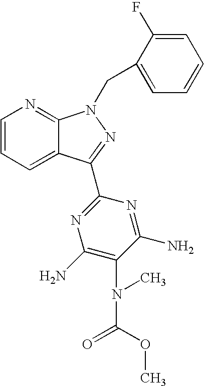
and salts, isomers and hydrates thereof.

The compounds of the formula (I) may also exist in the form of their salts. In general, mention may be made here of salts with organic or inorganic bases or acids.

The compounds of the formula (I) may exist in tautomeric forms. This is known to the skilled worker, and such forms are likewise encompassed by the invention.

The compounds of the formula (I) may also occur in the form of their possible hydrates.

The compounds of the formula (I) can be prepared for example
[A] by reacting compounds of the formula (Ia) 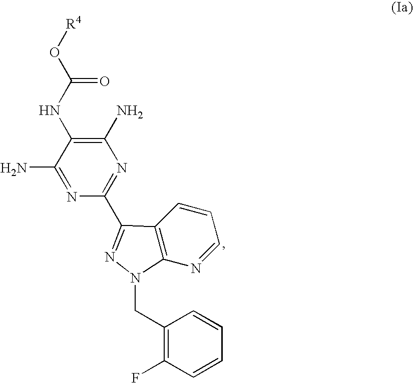
in which
R⁴ is as defined above,
with compounds of the formula (II)
   R³—X¹ (II),
in which
R³ is as defined above, and
X¹ is a leaving group such as, for example, halogen, preferably iodine, or mesylate,
where appropriate in an organic solvents with cooling to give compounds of the formula (I) or
[B] by reacting the compound of the formula (II) 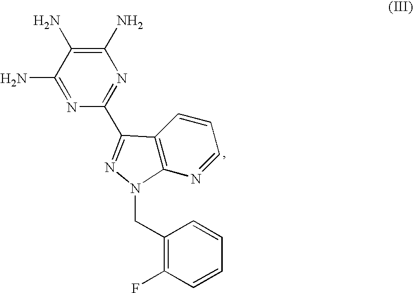
with compounds of the formula (IV) 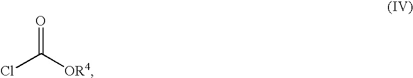
in which
R⁴ is as defined above,
where appropriate in an organic solvent to give compounds of the formula (Ia),
   or
   [C] by reacting the compound of the formula (V) 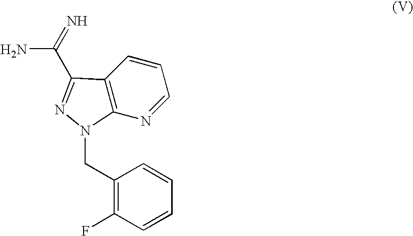
with compounds of the formula (VI) 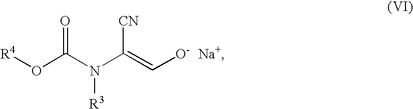
in which
R³ and R⁴ are as defined above,
where appropriate in an organic solvent with heating to give compounds of the formula (Ib) 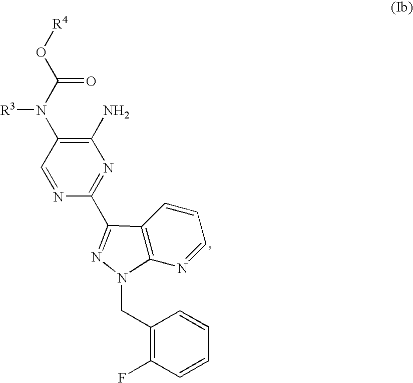
in which
R³ and R⁴ are as defined above.

For the purposes of the present invention, halogen stands for fluorine, chlorine, bromine and iodine.

The compounds of the formula (II) and (IV) are commercially available, disclosed in the literature or can be prepared in a manner known to the skilled worker.

The compound of the formula (III) can be prepared as shown in the following reaction scheme:

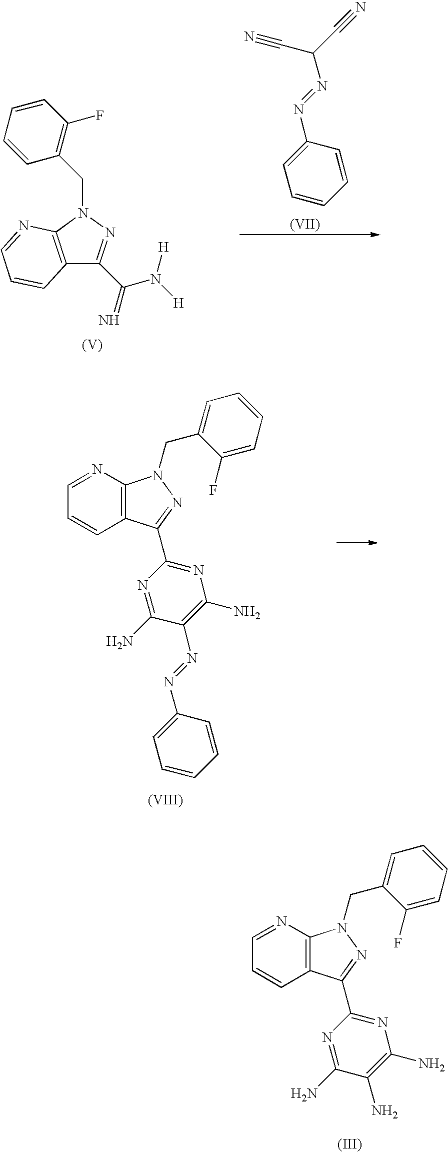

Compound (III) can be obtained in a two-stage synthesis by reacting compound (V) with compound (VII) to give compound (VIII) in accordance with process step [C] and subsequent hydrogenation of the compound (VIII) with aqueous Raney nickel. The hydrogenation can be carried out in an organic solvent, for example dimethylformamide, preferably under elevated pressure, for example from 50 to 70 bar, preferably at 65 bar, stirring the reaction solution for several hours, for example for 22 hours, at elevated temperature, for example at from to 40 to 80° C., preferably at from 60° C. to 65° C.

The compound (VII) can be prepared in analogy to L. F. Cavalieri, J. F. Tanker, A. Bendich, J. Am. Chem. Soc., 1949, 71, 533.

The compound (V) can be prepared as shown in the following reaction scheme:

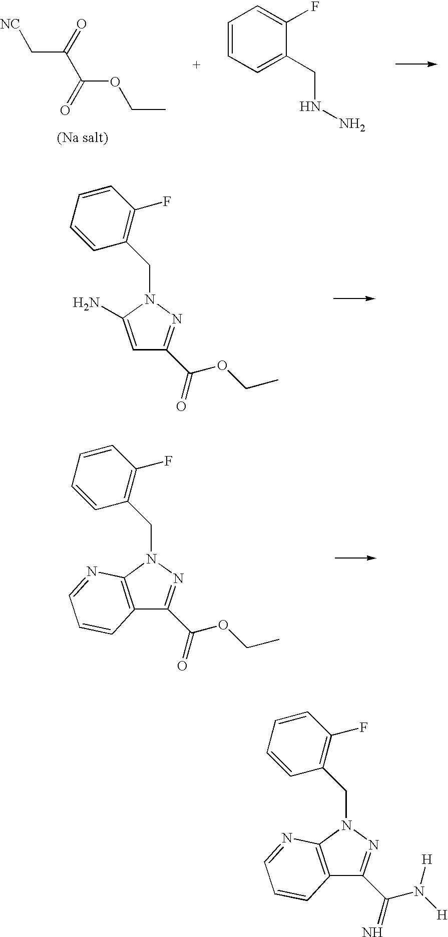

Compound (V) can be obtained in a multistage synthesis from the sodium salt of ethyl cyanopyruvate, which is known from the literature (Borsche and Manteuffel, Liebigs. Ann. Chem. 1934, 512, 97). Reaction thereof with 2-fluorobenzylhydrazine with heating under a protective gas atmosphere in an inert solvent such as dioxane results in ethyl 5-amino-1-(2-fluorobenzyl)pyrazole-3-carboxylate, which can be cyclized to give the corresponding pyridine derivative by reaction with dimethylaminoacrolein in acidic medium under a protective gas atmosphere with heating. This pyridine derivative, ethyl 1-(2-fluorobenzyl)-1H-pyrazolo[3,4-b]pyridine-3-carboxylate, is converted by a multistage sequence consisting of conversion of the ester with ammonia into the corresponding amide, dehydration with a dehydrating agent such as trifluoroacetic anhydride to the corresponding nitrile derivative, reaction of the nitrile derivative with sodium ethoxide and final reaction with ammonium chloride into the compound (V).

The compounds of the formula (VI) can be synthesized by methods known to the skilled worker from the corresponding carbamates by reaction with ethyl formate. The carbamates can be prepared in analogy to Q. Li. Chu, T. W. Daniel, A. Claiborne, C. S. Cooper, C. M. Lee, J. Med. Chem. 39 (1996) 3070-3088.

Reaction of the compounds of the formulae (Ia) and (II) to give compounds of the formula (I) can be carried out by employing the reactants in equimolar amounts in an organic solvent, for example dimethylformamide or tetrahydrofuran, preferably in the presence of from 1 to 2 equivalents, preferably 1.1 to 1.5 equivalents, of a base such as, for example sodium hydride or sodium N,N-bistrimethylsilylamide, preferably under atmospheric pressure and with stirring of the reaction for a few hours, for example for 1 hour, while cooling, for example at −10° C. to room temperature, preferably at 0° C.

Reaction of the compounds of the formulae (III) and (IV) to give the compounds of the formula (Ia) can be carried out by using the reactants in equimolar amounts in an organic solvent, for example an organic base, preferably pyridine, preferably under atmospheric pressure and with stirring of the reaction solution for several hours, for example for 12 hours, at 0° C. to room temperature, preferably at room temperature.

Reaction of compounds of the formulae (V) and (VI) to give compounds of the formula (Ib), or of compounds of the formulae (V) and (VII) to give compounds of the formula (VIII), can be carried out by using the reactants in equimolar amounts or with use of the compound of the formula (VI) in slight excess in an organic solvent such as, for example, in a hydrocarbon such as toluene or xylene or in N,N-dimethylformamide, preferably in the presence of 2-3 equivalents, preferably 2 equivalents, of a base such as, for example triethylamine or sodium methanolate, preferably under atmospheric pressure and with stirring of the reaction solution for several hours, for example for 9 hours, at elevated temperature, for example at 80-160° C., preferably at 100-150° C., in particular at 110° C.

The present invention further relates to the use of lipid-lowering agents for enhancing the effect of direct soluble guanylate cyclase stimulators of the formula (I) in the treatment of diseases which can be influenced by stimulating soluble guanylate cyclase.

Preferred examples which may be mentioned are: cardiovascular disorders such as hypertension or heart failure, stable and unstable angina pectoris, peripheral and cardiac vascular disorders, arrhythmias, thromboembolic disorders and ischemias such as myocardial infarction, stroke, transistorily, and ischemic attacks, disturbances of peripheral blood flow, prevention of restenoses as after thrombolysis therapies, percutaneously transluminal angioplasties (PTA), percutaneously transluminal coronary angioplasties (PTCA), bypass, and arteriosclerosis, asthmatic disorders and diseases of the urogenital systems such as prostate hypertrophy, erectile dysfunction, female sexual dysfunction, osteoporosis, glaucoma, pulmonary hypertension, gastroparesis or incontinence.

Mention may further be made of the control of central nervous system diseases characterized by disturbances of the NO/cGMP system: improvement of perception, concentration, learning or memory after cognitive impairments like those occurring in particular in situations/disorders/syndromes such as mild cognitive impairment, age-associated learning and memory impairments, age-associated memory losses, vascular dementia, craniocerebral trauma, stroke, dementia occurring after strokes, post-traumatic craniocerebral trauma, general concentration impairments, concentration impairments in children with learning and memory problems, Alzheimer's disease, Lewy body dementia, dementia with degeneration of the frontal lobes including Pick's syndrome, Parkinson's disease, progressive nuclear palsy, dementia with corticobasal degeneration, amyolateral-sclerosis (ALS), Huntington's disease, multiple sclerosis, thalamic degeneration, Creutzfeld-Jacob dementia, HIV dementia, schizophrenia with dementia or Korsakoff's psychosis; states of anxiety, tension and depression, CNS-related sexual dysfunctions and sleep impairments; regulation of pathological disturbances of the intake of food, stimulants and addictive substances; regulation of cerebral blood flow and control of migraine; prophylaxis and control of the sequelae of cerebral infarction such as stroke, cerebral ischemias and of craniocerebral trauma; control of states of pain or as antiinflammatory agents.

Apart from the two active ingredient components A and B mentioned above, the combination product of the invention may also comprise any other active ingredients as long as they do not conflict with the area of indications and do not impair the effect of the direct soluble guanylate cyclase stimulator of the formula (I) and of the lipid-lowering agent. In particular, it is possible to add to the composition of the invention organic nitrates or NO donors—that is to say compounds which stimulate the synthesis of cGMP—or compounds which inhibit the breakdown of cyclic guanosine monophosphate (cGMP).

Organic nitrates and NO donors for the purposes of the invention are generally substances which display their therapeutic effect via release of NO or NO species. Sodium nitroprusside, nitroglycerine, isosorbide dinitrate, isosorbide mononitrate, molsidomine and SIN-1 are preferred.

The invention additionally encompasses combination with compounds which inhibit the breakdown of cyclic guanosine monophosphate (cGMP). These are, in particular, inhibitors of phosphodiesterases 1, 2 and 5; nomenclature of Beavo and Reifsnyder (1990) TiPS 11 pages 150 to 155. These inhibitors potentiate the effect of the compound of the invention and increase the desired pharmacological effect.

These other active ingredients which are preferably present may—just like the active ingredient components A and B—be present either as true mixture together with A and/or B or else be present spatially separate therefrom. Administration thereof can take place in parallel or simultaneously or sequentially in relation to the active ingredient component(s) A and/or B.

The other active ingredients preferably present in the combination product of the invention include, for example:
other active ingredients improving erectile ability, for example: cGMP PDE inhibitors such as, for example, sildenafil (EP-B-0 463 756), IC 351 (WO 95/19978) or vardenafil (WO 99/24433), α-adrenergic antagonists such as, for example, yohimbine or Vasomax® from Zonagen; or else substances like those mentioned in WO-A-98/52569, the contents of which are hereby included by reference; or prostaglandins E1; or seretonin antagonists;
active ingredients from the cardiovascular area of indications;
active ingredients from the CNS and cerebral areas of indications;
vitamins;
minerals;
trace elements.

All conventional administration forms are suitable in each case for administering the two active ingredient components A and B (and the other active ingredients present where appropriate). Administration preferably takes place orally, perlingually, sublingually, nasally, transdermally, buccally, intravenously, rectally, by inhalation or parenterally. Administration preferably takes place orally, sublingually or nasally. Oral administration is very particularly preferred.

It is additionally possible to administer the two active ingredient components A and B in different dosage forms if administration is spatially separate or at different times.

The two active ingredient components A and B can be converted—together or spatially separate—in each case in a manner known per se into the conventional formulations such as tablets, coated tablets, pills, granules, aerosols, syrups, emulsions, suspensions and solutions, using inert, nontoxic, pharmaceutically suitable carriers or solvents. In these cases, the therapeutically active components A and B should each be present in a concentration of about 0.5 to 90% by weight of the complete mixture, i.e. in amounts which suffice to reach the stated dosage range.

The formulations are produced for example by extending the two active ingredient components A and B with solvents and/or carriers, where appropriate using emulsifiers and/or dispersants, it being possible, for example in the case where water is used as diluent, where appropriate to use organic solvents as auxiliary solvents.

The present invention further relates to a process for producing the composition of the invention, characterized in that at least one lipid-lowering agent and at least one direct soluble guanylate cyclase stimulator of the formula (I) is converted, where appropriate with conventional excipients and additives, into a suitable administration form.

The dosages administered on oral administration for human use are from 0.001 to 50 mg/kg, preferably from 0.001 mg/kg to 20 mg/kg, in particular 0.001 to 10 mg/kg, of body weight, particularly preferably 0.001 mg/kg to 5 mg/kg, of the respective active ingredient component A or B, to achieve effective and worthwhile results.

It may nevertheless be necessary where appropriate to depart from the amounts mentioned here, in particular depending on the body weight and the nature of the administration route, or on the individual behavior towards the combination product, on the nature of the formulation and on the time or interval over which administration takes place. Thus, it may be sufficient in some cases to make do with less than the aforementioned minimum amount while in other cases the upper limit mentioned must be exceeded.

It may be advisable in the case where relatively large amounts are administered for these to be distributed in a plurality of single doses over the day.

### EXPERIMENTAL SECTION

Abbreviations:

ACN acetonitrile
BABA n-butyl acetate/n-butanol/glacial acetic acid/phosphate buffer pH 6 (50:9:25.15; org. phase)
conc. concentrated
DCI direct chemical ionization (in MS)
DCM dichloromethane
DIEA N,N-diisopropylethylamine
DMSO dimethyl sulfoxide
DMF N,N-dimethylformamide
EA ethyl acetate
EI electron impact ionization (in MS)
ESI electrospray ionization (in MS)
h hour
HPLC high pressure, high performance liquid chromatography
LC-MS coupled liquid chromatography/mass spectroscopy
LDA lithium diisopropylamide
MCPBA m-chloroperoxybenzoic acid
m.p. melting point
MS mass spectroscopy
NMR nuclear magnetic resonance spectroscopy
R_{f} retention index (in TLC)
RP-HPLC reverse phase HPLC
RT room temperature
Rₜ retention time (in HPLC)
sat. saturated
THF tetrahydrofuran
TLC thin layer chromatography

Mobile Phases for Thin Layer Chromatography:

| | |
|---|---|
| T1 E1: | toluene/ethyl acetate (1:1) |
| T1 EtOH1: | toluene/ethanol (1:1) |
| C1 E1: | cyclohexane/ethyl acetate (1:1) |
| C1 E2: | cyclohexane/ethyl acetate (1:2) |

LCMS and HPLC Methods:
Method 1 (LCMS)

Instrument: Micromass Platform LCZ, HP1100; column: Symmetry C18, 50 mm×2.1 mm, 3.5 μm; eluent A: acetonitrile+0.1% formic acid, eluent B: water+0.1% formic acid; gradient: 0.0 min 10% A→4.0 min 90% A→6.0 min 90% A; oven: 40° C.; flow rate: 0.5 ml/min; UV detection: 208-400 nm.

Method 2 (LCMS)

Instrument: Micromass Quattro LCZ, HP1100; column: Symmetry C18, 50 mm×2.1 mm, 3.5 μm; eluent A: acetonitrile+0.1% formic acid, eluent B: water+0.1% formic acid; gradient: 0.0 min 10% A→4.0 min 90% A→6.0 min 90% A; oven: 40° C.; flow rate: 0.5 ml/min; UV detection: 208-400 nm.

Method 3 (LCMS)

Instrument: Waters Alliance 2790 LC; column: Symmetry C18, 50 mm×2.1 mm, 3.5 μm; eluent A: water+0.1% formic acid, eluent B: acetonitrile+0.1% formic acid; gradient: 0.0 min 5% B→5.0 min 10% B→6.0 min 10% B; temperature: 50° C.; flow rate: 1.0 ml/min; UV detection: 210 nm.

Method 4 (HPLC)

Instrument: HP 1100 with DAD detection; column: Kromasil RP-18, 60 mm×2 mm, 3.5 μm; eluent: A=5 ml of HClO₄/l of H₂O, B=ACN; gradient: 0 min 2% B, 0.5 min 2% B, 4.5 min 90% B, 6.5 min 90% B; flow rate: 0.75 ml/min; temp.: 30° C.; detection UV 210 nm.

Preparative RP-HPLC

Column: YMC gel; eluent: acetonitrile/water (gradient); flow rate: 50 ml/min; temp.: 25° C.; detection UV 210 nm.

Starting Compounds:

### Example 1A

### Ethyl 5-amino-1-(2-fluorobenzyl)pyrazole-3-carboxylate

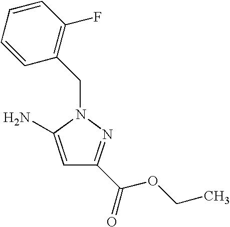

111.75 g (75 ml, 0.98 mol) of trifluoroacetic acid are added to 100 g (0.613 mol) of sodium salt of ethyl cyanopyruvate (prepared in analogy to Borsche and Manteuffel, Liebigs Ann. 1934, 512, 97) in 2.5 l of dioxane under argon with efficient stirring at room temperature, and the mixture is stirred for 10 minutes during which much of the precursor dissolves. Then 85.93 g (0.613 mol) of 2-fluorobenzylhydrazine are added, and the mixture is boiled overnight. After cooling, the crystals of sodium trifluoroacetate which have separated out are filtered off with suction and washed with dioxane, and the solution is reacted further as it is.

### Example 2A

### Ethyl 1-(2-fluorobenzyl)-1H-pyrazolo[3,4-b]pyridine-3-carboxylate

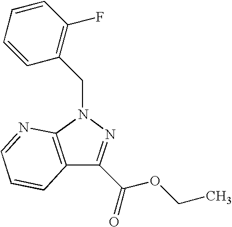

The solution obtained from Example 1A is mixed with 61.25 ml (60.77 g, 0.613 mol) of dimethylaminoacrolein and 56.28 ml (83.88 g, 0.736 mol) of trifluoroacetic acid and boiled under argon for 3 days. The solvent is then evaporated in vacuo, and the residue is added to 2 l of water and extracted three times with 1 l of ethyl acetate each time. The combined organic phases are dried with magnesium sulfate and concentrated in a rotary evaporator. Chromatography is carried out on 2.5 kg of silica gel, eluting with a toluene/toluene-ethyl acetate=4:1 gradient. Yield: 91.6 g (49.9% of theory over two stages).

Melting point 85° C.

R_{f} (SiO₂, T1 E1): 0.83

### Example 3A

### 1-(2-Fluorobenzyl)-1H-pyrazolo[3,4-b]pyridine-3-carboxamide

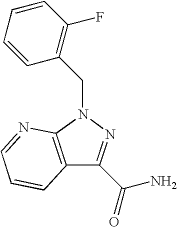

10.18 g (34 mmol) of the ester obtained in Example 2A are introduced into 150 ml of methanol which has been saturated with ammonia at 0-10° C. The mixture is stirred at room temperature for two days and then concentrated in vacuo.

R_{f} (SiO₂, T1 E1): 0.33

### Example 4A

### 3-Cyano-1-(2-fluorobenzyl)-1H-pyrazolo[3,4-b]pyridine

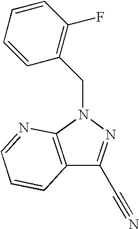

36.1 g (133 mmol) of 1-(2-fluorobenzyl)-1H-pyrazolo[3,4-b]pyridine-3-carboxamide from Example 3A are dissolved in 330 ml of THF, and 27 g (341 mmol) of pyridine are added. Then, over the course of 10 minutes, 47.76 ml (71.66 g, 341 mmol) of trifluoroacetic anhydride are added, during which the temperature rises to 40° C. The mixture is stirred at room temperature overnight. It is then added to 1 l of water and extracted three times with 0.5 l of ethyl acetate each time. The organic phase is washed with saturated sodium bicarbonate solution and with 1N hydrochloric acid, dried with magnesium sulfate and concentrated in a rotary evaporator.

Yield: 33.7 g (100% of theory)

Melting point: 81° C.

R_{f} (SiO₂, T1 E1): 0.74

### Example 5A

### Methyl (2-fluorobenzyl)-1H-pyrazolo[3,4-b]pyridine-3-carboximidate

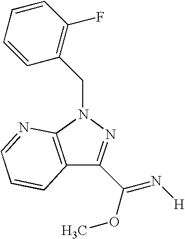

30.37 g (562 mmol) of sodium methoxide are dissolved in 1.5 l of methanol, and 36.45 g (144.5 mmol) of 3-cyano-1-(2-fluorobenzyl)-1H-pyrazolo[3,4-b]pyridine (from Example 4A) are added. The mixture is stirred at room temperature for 2 hours and the resulting solution is employed directly for the next stage.

### Example 6A

### 1-(2-Fluorobenzyl)1H-pyrazolo[3,4-b]pyridine-3-carboxamidine

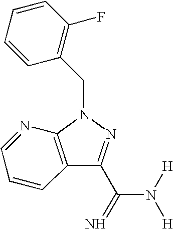

The solution of methyl (2-fluorobenzyl)-1H-pyrazolo[3,4-b]pyridine-3-carboximidate in methanol obtained from Example 5A is mixed with 33.76 g (32.19 ml, 562 mmol) of glacial acetic acid and 9.28 g (173 mmol) of ammonium chloride and stirred under reflux overnight. The solvent is evaporated in vacuo, the residue is thoroughly triturated with acetone, and the precipitated solid is filtered off with suction. It is added to 2 l of water, 31.8 g of sodium carbonate are added while stirring, the mixture is extracted three times with a total of 1 l of ethyl acetate, and the organic phase is dried with magnesium sulfate and evaporated in vacuo.

Yield 27.5 g (76.4% of theory over two stages)

m.p.: 86° C.

R_{f} (SiO₂, T1 EtOH1): 0.08

### Example 7A

### 2-[1-(2-Fluorobenzyl)-1H-pyrazolo[3,4-b]pyridin-3-yl]-5-[(E)phenyldiazenyl]-4,6-pyrimidinediamine

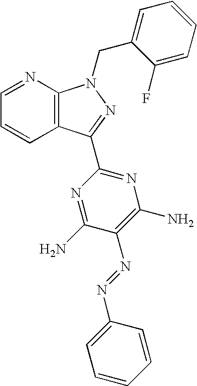

3.87 g of sodium methanolate and then 12.2 g (71.7 mmol) of phenylazomalononitrile (L. F. Cavalieri, J. F. Tanker, A. Bendich, J. Am. Chem. Soc., 1949, 71, 533) are added to a stirred solution of 21.92 g (71.7 mmol) of 1-(2-fluorobenzyl)-1H-pyrazolo[3,4-b]pyridine-3-carboxamidine in N,N-dimethylformamide from Example 6A. The mixture is stirred at 110° C. overnight and allowed to cool. The solid which precipitates is filtered off with suction and washed with ethanol. Drying results in 23 g (73% of theory) of the target compound.

### Example 8A

### 2-[1-(2-Fluorobenzyl)-1H-pyrazolo[3,4-b]pyridin-3-yl]-4,5,6-pyrimidinetriamine trihydrochloride

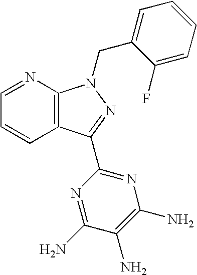

5 g (11.38 mmol) of 2-[1-(2-fluorobenzyl)-1H-pyrazolo[3,4-b]pyridin-3-yl]-5-[(E)-phenyldiazenyl]-4,6-pyrimidinediamine from Example 7A are hydrogenated with 800 mg of 50% Raney nickel in water in 60 ml of DMF under a pressure of 65 bar of hydrogen at 62° C. for 22 hours. The catalyst is filtered off with suction through kieselguhr, and the solution is evaporated in vacuo and stirred with 5N hydrochloric acid. The yellowish brown precipitate which separates out is filtered off with suction and dried. 3.1 g (59.3% of theory) of the target compound are obtained. The free base is obtained by shaking with dilute sodium bicarbonate solution and extracted with ethyl acetate. The solid insoluble in both phases is filtered off with suction. The ethyl acetate phase also contains small amounts of the free base.

### Example 9A

### Methyl cyanomethyl(methyl)carbamate

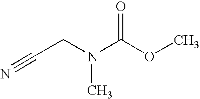 prepared in analogy to: Q. Li. Chu, T. W. Daniel, A. Claiborne, C. S. Cooper, C. M. Lee, J. Med. Chem. 1996, 39, 3070-3088.

### Example 10A

### Sodium (E)-2-cyano-2-[(methoxycarbonyl)(methyl)amino]ethenolate

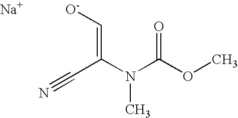

0.46 g (0.01 mmol) of sodium methoxide is added under argon to tetrahydrofuran (solution A). Then 1.00 g (0.01 mmol) of methyl cyanomethyl(methyl)carbamate from Example 9A is added to 1.73 g (0.02 mmol) of ethyl formate. Solution A is slowly and carefully added dropwise to this mixture. The mixture is stirred at RT overnight. The solvent is concentrated in vacuo in a rotary evaporator, and diethyl ether is added to the residue. The resulting crystals are filtered off with suction and dried under high vacuum.

Yield: 1.05 g (76% of theory)

HPLC (Method 4): Rₜ=1.35 min.

¹H-NMR (200 MHz, DMSO-d₆): δ=2.90 (d, 1H), 3.35 (s, 3H), 3.47 (s, 3H).

### EXAMPLES

### Example 1

### Ethyl 4-amino-2-[1-(2-fluorobenzyl)-1H-pyrazolo[3,4-b]pyridin-3-yl]-5-pyrimidinyl(methyl)carbamate

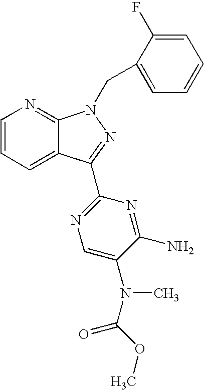

Under argon, 0.80 g (2.61 mmol) of 1-(2-fluorobenzyl)-1H-pyrazolo[3,4-b]pyridine-3-carboxamidine from Example 6A, 0.51 g (2.86 mmol) of sodium (E)-2-cyano-2-[(methoxycarbonyl)(methyl)amino]ethenolate from Example 10A and 0.53 g (0.73 ml, 5.23 mmol) of triethylamine are added to 50 ml of toluene. The mixture is boiled to reflux for 9 hours. It is then cooled to RT again and is mixed and extracted with dichloromethane and water. The organic phase is dried over magnesium sulfate, filtered and concentrated in vacuo in a rotary evaporator. The residue is mixed with 5 ml of diethyl ether and crystallizes therewith. The crystals are filtered off with suction, dried and purified by preparative RP-HPLC.

Yield: 20.2 mg (2% of theory)

LC/MS (Method 2): Rₜ=3.01 min

MS (EI): m/z=408 (M+H)⁺

¹H-NMR (300 MHz, DMSO-d₆): δ=3.09 (s, 3H), 3.29 (s, 3H), 5.83 (s, 2H), 7.09-7.42 (m, 5H), 8.20 (s, 1H), 8.64 (dd, 1H). 8.94 (dd, 1H), 9.27 (br. s, 2H).

### Example 2

### Ethyl 4,6-diamino-2-[1-(2-fluorobenzyl)-1H-pyrazolo[3,4-b]pyridin-3-yl]-5-pyrimidinylcarbamate

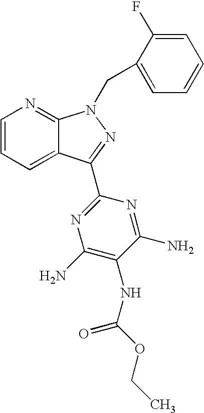

107.35 mg (0.31 mmol) of 2-[1-(2-fluorobenzyl)-1H-pyrazolo[3,4-b]pyridin-3-yl]-4,5,6-pyrimidinetriamine trihydrochloride from Example 8A are added to 5 ml of pyridine, and the mixture is cooled to 0° C. 33.25 mg (0.31 mmol) of ethyl chloroformate are added, and the reaction is left to stir at RT overnight. The pyridine is evaporated in vacuo in a rotary evaporator, and the residue is purified by preparative RP-HPLC.

Yield: 56.2 mg (43% of theory)

LC/MS (Method 1): Rₜ=2.66 min

MS (EI): m/z=423 (M+H)⁺

¹H-NMR (300 MHz, DMSO-d₆): δ=1.17-1.33 (m, 3H), 3.97-4.14 (m, 2H), 5.80 (s, 2H), 6.14 (br. s, 4H), 7.07-7.17 (m, 2H), 7.22 (t, 1H). 7.29-7.40 (m, 2H), 7.97 (br. s, 1H), 8.60 (d, 1H), 9.07 (d, 1H).

### Example 3

### Isopropyl 4,6-diamino-2-[1-(2-fluorobenzyl)-1H-pyrazolo[3,4-b]pyridin-3-yl]-5-pyrimidinylcarbamate

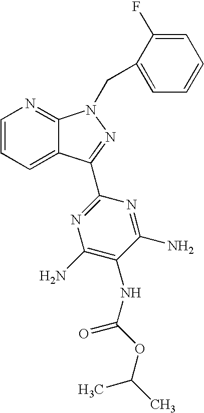

Prepared in analogy to Example 2 with 150 mg (0.43 mmol) of 2-[1-(2-fluorobenzyl)-1H-pyrazolo[3,4-b]pyridin-3-yl]-4,5,6-pyrimidinetriamine trihydrochloride from Example 8A, 7.5 ml of pyridine and 52.47 mg (0.43 mmol) of isopropyl chloroformate. The residue is taken up in a dichloromethane/methanol mixture, filtered and dried.

Yield: 165 mg (88% of theory)

LC/MS (Method 1): Rₜ=2.84 min

MS (EI): m/z=437 (M+H)⁺

¹H-NMR (300 MHz, DMSO-d₆): δ=1.26 (d, 6H), 4.82 (quin., 1H), 5.92 (s, 2H), 7.07-7.20 (m, 2H), 7.25 (t, 1H). 7.31-7.43 (m, 2H), 7.47-7.57 (m, 1H), 8.16 (br. s, 1H), 8.74 (dd, 1H), 8.98 (dd, 1H).

### Example 4

### Neopentyl 4,6-diamino-2-[1-(2-fluorobenzyl)-1H-pyrazolo[3,4-b]pyridin-3-yl]-5-pyrimidinylcarbamate

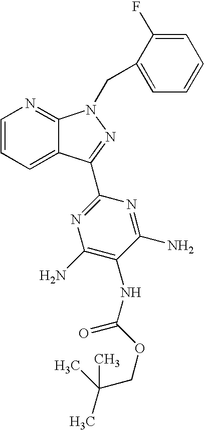

Prepared in analogy to Example 2 with 100 mg (0.29 mmol) of 2-[1-(2-fluorobenzyl)-1H-pyrazolo[3,4-b]pyridin-3-yl]-4,5,6-pyrimidinetriamine trihydrochloride from Example 8A, 5 ml of pyridine and 43 mg (0.29 mmol) of neopentyl chlorocarbonate.

Yield: 54 mg (41% of theory)

LC/MS (Method 1): Rₜ=3.10 min

MS (EI): m/z=465 (M+H)⁺

¹H-NMR (400 MHz, DMSO-d₆): δ=0.95 (br. s, 9H), 3.74 (s, 2H), 5.79 (s, 2H), 6.10 (br. s, 4H), 7.08-7.17 (m, 2H), 7.22 (t, 1H), 7.29-7.39 (m, 2H), 8.00 (br. s, 1H), 8.60 (dd, 1H), 9.06 (dd, 1H).

### Example 5

### Methyl 4,6-diamino-2-[1-(2-fluorobenzyl)-1H-pyrazolo[3,4-b]pyridin-3-yl]-5-pyrimidinylcarbamate

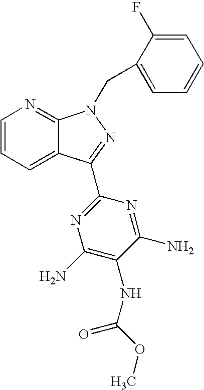

30.5 g (87.0 mmol) of 2-[1-(2-fluorobenzyl)-1H-pyrazolo[3,4-b]pyridin-3-yl]-4,5,6-pyrimidinetriamine trihydrochloride from Example 8A are dissolved in 30 ml of pyridine. The resulting solution is cooled to 0° C. 8.22 g (87.0 mmol) of methyl chloroformate are added, and the mixture is stirred at 0° C. for a further 2 hours. It is then allowed to warm to room temperature and is stirred for a further 2 hours. The residue after concentration in vacuo is washed with water and dried. It is further purified by stirring in 300 ml of boiling diethyl ether. The precipitated product is filtered off with suction and dried in vacuo.

Yield: 32.6 g (92% of theory)

LC/MS (Method 1): Rₜ=2.61 min

MS (EI): m/z=409 (M+H)⁺

¹H-NMR (400 MHz, DMSO-d₆): δ=3.61 (s, 3H), 5.80 (s, 2H), 6.19 (br. s, 4H), 7.08-7.16 (m, 2H), 7.22 (t, 1H), 7.28-7.39 (m, 2H), 7.99 (br. s, 1H), 8.60 (dd, 1H), 9.05 (dd, 1H).

### Example 6

### Ethyl 4,6-diamino-2-[1-(2-fluorobenzyl)-1H-pyrazolo[3,4-b]pyridin-3-yl]-5-pyrimidinyl(methyl)carbamate

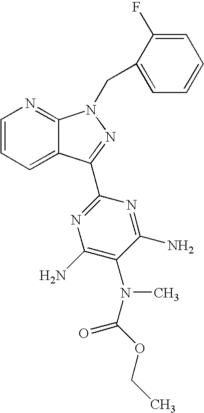

54 mg (0.13 mmol) of ethyl 4,6-diamino-2-[1-(2-fluorobenzyl)-1H-pyrazolo[3,4-b]pyridin-3-yl]-5-pyrimidinylcarbamate from Example 3 are added to 5 ml of DMF, the mixture is cooled to 0° C., and 7.67 mg (0.19 mmol) of sodium hydride are added. Then 18.14 mg (0.13 mmol) of iodomethane are added dropwise, and the mixture is left to stir for one hour. The mixture is mixed with water and extracted with dichloromethane. The combined organic phases are dried over magnesium sulfate and concentrated in a rotary evaporator. The residue is purified first by column chromatography (mobile phase: dichloromethane/methanol=10:1) and then by preparative RP-HPLC.

Yield: 32 mg (58% of theory)

LC/MS (Method 2): Rₜ=2.91 min

MS (EI): m/z=437 (M+H)⁺

¹H-NMR (200 MHz, DMSO-d₆): δ=1.08 (t, 3H), 2.99 (s, 3H), 2.93-4.11 (m, 2H), 5.79 (s, 2H), 6.35 (br. s, 4H), 7.06-7.14 (m, 2H), 7.16-7.28 (m, 1H), 7.28-7.32 (m, 2H), 8.59 (dd, 1H), 9.06 (dd, 1H).

### Example 7

### Isopropyl 4,6-diamino-2-[1-(2-fluorobenzyl)-1H-pyrazolo[3,4-b]pyridin-3-yl]-5-pyrimidinyl(methyl)carbamate

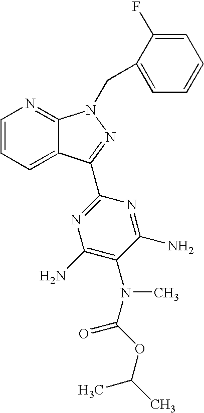

Prepared in analogy to Example 6 with 75 mg (0.17 mmol) of isopropyl 4,6-diamino-2-[1-(2-fluorobenzyl)-1H-pyrazolo[3,4-b]pyridin-3-yl]-5-pyrimidinylcarbamate from Example 3, 10.31 mg (0.26 mmol) of sodium hydride and 24.4 mg (0.17 mmol) of iodomethane. The residue is purified by preparative RP-HPLC.

Yield: 32 mg (41% of theory)

LC/MS (Method 1): Rₜ=2.97 min

MS (EI): m/z=451 (M+H)⁺

¹H-NMR (300 MHz, DMSO-d₆): δ=1.09 (d, 6H), 2.98 (s, 3H), 4.80 (quin., 1H), 5.79 (s, 2H), 6.31 (br. s, 4H), 7.05-7.16 (m, 2H), 7.22 (t, 1H), 7.28-7.40 (m, 2H), 8.59 (dd, 1H), 9.07 (dd, 1H).

### Example 8

### Methyl 4,6-diamino-2-[1-(2-fluorobenzyl)-1H-pyrazolo[3,4-b]pyridin-3-yl]-5-pyrimidinyl(methyl)carbamate

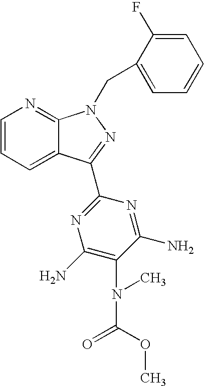

Prepared in analogy to Example 6 with 310 mg (0.76 mmol) of methyl 4,6-diamino-2-[1-(2-fluorobenzyl)-1H-pyrazolo[3,4-b]pyridin-3-yl]-5-pyrimidinylcarbamate from Example 5, 27.32 mg (1.14 mmol) of sodium hydride and 215.5 mg (1.52 mmol) of iodomethane. The mixture is worked up by adding water and 2 molar potassium hydroxide solution and extracting with dichloromethane. The combined organic phases are dried with magnesium sulfate and concentrated in a rotary evaporator. The residue is purified by preparative RP-HPLC.

Yield: 93 mg (29% of theory)

Larger amounts of the compound from Example 8 can also be prepared by the following synthetic method:

20.0 g (49.0 mmol) of methyl 4,6-diamino-2-[1-(2-fluorobenzyl)-1H-pyrazolo[3,4-b]pyridin-3-yl]-5-pyrimidinylcarbamate from Example 5 are dissolved in 257 ml of tetrahydrofuran and cooled to 0° C. 53.9 ml (49.0 mmol of a 1 M solution in tetrahydrofuran) of bis(trimethylsilyl)lithium amide are added dropwise over the course of 15 minutes. After stirring at 0° C. for 20 min, 6.95 g (53.9 mmol) of iodomethane are added. After one hour, the mixture is allowed to warm to room temperature and the reaction is stopped by adding saturated aqueous ammonium chloride solution. The phases are separated. The aqueous phase is extracted several times with ethyl acetate and dichloromethane. The combined organic phases are concentrated in vacuo. The residue obtained in this way is suspended in a mixture of dichloromethane and tetrahydrofuran (1:1). The insoluble crystals are filtered off with suction and taken up in methanol. The mixture is heated under reflux for one hour. After cooling, the precipitate which has separated out is filtered off. The red solid obtained in this way is suspended in 100 ml of a mixture of dioxane and dichloromethane (1:1) and, while boiling, 20 ml of methanol are added until a clear solution is formed. Activated carbon is added, and the mixture is briefly boiled and filtered hot through kieselguhr. The solution obtained in this way is evaporated to dryness. The residue is taken up in methanol, and the suspension is stirred at room temperature for one hour. The white crystals are filtered off with suction.

Yield: 14.9 g (72% of theory) LC/MS (method 3): Rₜ=1.85 min

MS (EI): m/z=423 (M+H)⁺

¹H-NMR (200 MHz, DMSO-d₆): δ=3.01 (s, 3H), 3.57 (s, 3H), 5.92 (s, 2H), 7.05.7.17 (m, 2H), 7.18-7.46 (m, 3H), 7.47-7.61 (m, 2H), 7.59-7.97 (m, 2H), 8.71-8.81 (m, 1H), 8.97 (dd, 1H).

### Example 9

### Isopropyl 4,6-diamino-2-[1-(2-fluorobenzyl)-1H-pyrazolo[3,4-b]pyridin-3-yl]-5-pyrimidinyl(ethyl)carbamate

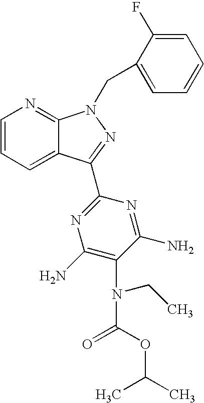

Prepared in analogy to Example 6 with 60 mg (0.14 mmol) of isopropyl 4,6-diamino-2-[1-(2-fluorobenzyl)-1H-pyrazolo[3,4-b]pyridin-3-yl]-5-pyrimidinylcarbamate from Example 3, 4.95 mg (0.21 mmol) of sodium hydride and 21.4 mg (0.17 mmol) of iodoethane. To complete the reaction, the same amount of sodium hydride and iodoethane are added once again. The residue is purified by preparative RP-HPLC.

Yield: 43 mg (67% of theory)

LC/MS (Method 1): Rₜ=2.97 min

MS (EI): m/z=465 (M+H)⁺

¹H-NMR (200 MHz, DMSO-d₆): δ=0.96-1.06 (m, 3H), 1.09 (d, 6H), 2.79-2.93 (m, 2H), 4.82 (quin., 1H), 5.80 (s, 2H), 6.25 (br. s, 4H), 7.01-7.14 (m, 2H), 7.15-7.50 (m, 3H), 8.60 (dd, 1H), 9.09 (dd, 1H).

1. A combination product comprising as pharmaceutically active ingredients at least one active ingredient component A and at least one active ingredient component B, wherein active ingredient component A is a direct stimulator of soluble guanylate cyclase having the formula (I) 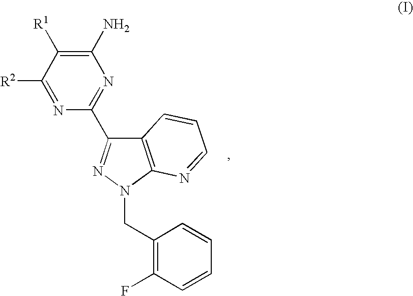
in which
R¹ is —NR³C(═O)OR⁴,
R² is hydrogen or NH₂,
R³ is hydrogen or (C₁-C₄)-alkyl,
R⁴ is (C₁-C₆)-alkyl,
and active ingredient component B is a lipid-lowering agent.

2. The combination product as claimed in claim 1,
wherein
R¹ is —NR³C(═O)OR⁴,
R² is NH₂,
R³ is methyl or ethyl, and
R⁴ is methyl, ethyl or isopropyl.

3. The combination product as claimed in claim 1, wherein the direct stimulator of soluble guanylate cyclase of the formula (I) has the following structure:

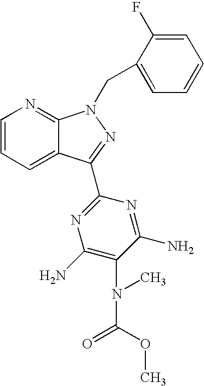

4. (canceled)

5. (canceled)

6. The combination product as claimed in any of claims 1 to 3, wherein the active ingredient components A and B are in the form of a functional unit.

7. The combination product as claimed in claim 1, wherein the active ingredient components A and B are (spatially) separate from one another, in the form of a kit of parts.

8. The combination product as claimed in claim 1, wherein the lipid-lowering agent (active ingredient component B) is selected from the group consisting of (a) HMG-CoA-reductase inhibitors; (b) squalene synthase inhibitors; (c) bile acid absorption inhibitors (bile acid sequestrants); (d) fibric acid and its derivatives; (e) nicotinic acid and its analogs; and (f) ω3-fatty acids.

9. The combination product as claimed in claim 8, wherein the lipid-lowering agent (active ingredient component B) is an HMG-CoA reductase inhibitor and is selected from the group of statins.

10. The combination product as claimed in claim 26, wherein the lipid-lowering agent (active ingredient component B) is atorvastatin or its salt, hydrate, alcoholate, ester or tautomer.

11. The combination product as claimed in claim 26, wherein the lipid-lowering agent (active ingredient component B) is cerivastatin or its salt, hydrate, alcoholate, ester and or tautomer.

12. A method for increasing the efficacy of direct soluble guanylate cyclase stimulators of the formula (I) as defined in claim 1 comprising administering a lipid-lowering agent in conjunction with said direct soluble guanylate cyclase stimulator of formula (I).

13. A process for producing compositions as claimed in claim 1, comprising converting at least one lipid-lowering agent and at least one direct soluble guanylate cyclase stimulator of the formula (I), where appropriate with conventional excipients and additives, into a suitable administration form.

14. A method for the treatment of cardiovascular disorders comprising administering an effective amount of a combination of a direct stimulator of soluble guanylate cyclase of formula (I) and a lipid-lowering agent as defined in claim 1, 2, 3, 6, 7, 8, 9, 10, or 11.

15. A method for the treatment of hypertension comprising administering an effective amount of a combination of a direct stimulator of soluble guanylate cyclase of formula (I) and a lipid-lowering agent as defined in claim 1, 2, 3, 6, 7, 8, 9, 10, or 11.

16. A method for the treatment of thromboembolic disorders and ischemias comprising administering an effective amount of a combination of a direct stimulator of soluble guanylate cyclase of formula (I) and a lipid-lowering agent as defined in claim 1, 2, 3, 6, 7, 8, 9, 10, or 11.

17. A method for the treatment of sexual dysfunction comprising administering an effective amount of a combination of a direct stimulator of soluble guanylate cyclase of formula (I) and a lipid-lowering agent as defined in claim 1, 2, 3, 6, 7, 8, 9, 10, or 11.

18. A method for the treatment of arteriosclerosis comprising administering an effective amount of a combination of a direct stimulator of soluble guanylate cyclase of formula (I) and a lipid-lowering agent as defined in claim 1, 2, 3, 6, 7, 8, 9, 10, or 11.

19. A method for the treatment of osteoporosis comprising administering an effective amount of a combination of a direct stimulator of soluble guanylate cyclase of formula (I) and a lipid-lowering agent as defined in claim 1, 2, 3, 6, 7, 8, 9, 10, or 11.

20. A method for the treatment of inflammation comprising administering an effective amount of a combination of a direct stimulator of soluble guanylate cyclase of formula (I) and a lipid-lowering agent as defined in claim 1, 2, 3, 6, 7, 8, 9, 10, or 11.

21. A method for the treatment of central nervous system disorders comprising administering an effective amount of a combination of a direct stimulator of soluble guanylate cyclase of formula (I) and a lipid-lowering agent as defined in claim 1, 2, 3, 6, 7, 8, 9, 10, or 11.

22. The method as claimed in any of claims 14 to 21, where the direct stimulator of soluble guanylate cyclase of formula (I) and the lipid-lowering agent are employed in combination with organic nitrates or NO donors or in combination with compounds which inhibit the breakdown of cyclic guanosine monophosphate (cGMP).

23. The combination product as claimed in claim 6 wherein said functional unit is a mixture, mix, or blend.

24. The combination product of claim 7 wherein the separation of active ingredient components A and B permits them to be separately administered either simultaneously or sequentially.

25. The combination product of claim 24 wherein the separation of active ingredient components A and B permits them to be separately administered sequentially.

26. The combination product as claimed in claim 8, wherein the lipid-lowering agent is selected from the group of statins consisting of atorvastatin, cerivastatin, fluvastatin, lovastatin, pravastatin, pitavastatin, simvastatin and rosuvastatin, and their respective salts, hydrates, alcohols, esters and tautomers
